(19) United States
(12) Patent Application Publication
(10) Pub. No.: US 2023/0363908 A1
(43) Pub. Date: Nov. 16, 2023

(54) SUTURE ANCHOR FOR MINIMALLY INVASIVE HEART VALVE REPAIR

(71) Applicant: NeoChord, Inc., St. Louis Park, MN (US)

(72) Inventors: Andy Freeman, St. Louis Park, MN (US); Daryl Edmiston, Draper, UT (US); Tyler Nordmann, Maple Grove, MN (US)

(21) Appl. No.: 18/197,260

(22) Filed: May 15, 2023

Related U.S. Application Data
(60) Provisional application No. 63/341,697, filed on May 13, 2022.

(51) Int. Cl. A61F 2/24

(52) U.S. Cl. CPC A61F 2/2457; A61F 2220/0016

(57) ABSTRACT

Disclosed herein are embodiments of cardiac anchors configured to be inserted into a heart wall of a patient to anchor a suture as an artificial chordae under an appropriate tension for proper valve function. Such cardiac anchors are particularly suitable for use in intravascular, transcatheter procedures.

### RELATED APPLICATION

This application claims the benefit of U.S. Provisional Application No. 63/341,697 filed May 13, 2022, which is hereby fully incorporated herein by reference.

### TECHNICAL FIELD

The present invention relates to minimally invasive delivery of a suture into the heart. More particularly, the disclosed embodiments relate to inserting and anchoring one or more sutures as artificial chordae tendineae for a flailing or prolapsing leaflet in a beating heart.

### BACKGROUND

The mitral and tricuspid valves inside the human heart include an orifice (annulus), two (for the mitral) or three (for the tricuspid) leaflets and a subvalvular apparatus. The subvalvular apparatus includes multiple chordae tendineae, which connect the mobile valve leaflets to muscular structures (papillary muscles) inside the ventricles. Rupture or elongation of the chordae tendineae results in partial or generalized leaflet prolapse, which causes mitral (or tricuspid) valve regurgitation. A commonly used technique to surgically correct mitral valve regurgitation is the implantation of artificial chordae (usually 4-0 or 5-0 Gore-Tex sutures) between the prolapsing segment of the valve and the papillary muscle.

This technique for implantation of artificial chordae was traditionally done by an open heart operation generally carried out through a median sternotomy and requiring cardiopulmonary bypass with aortic cross-clamp and cardioplegic arrest of the heart. Using such open heart techniques, the large opening provided by a median sternotomy or right thoracotomy enables the surgeon to see the mitral valve directly through the left atriotomy, and to position his or her hands within the thoracic cavity in close proximity to the exterior of the heart for manipulation of surgical instruments, removal of excised tissue, and/or introduction of an artificial chordae through the atriotomy for attachment within the heart. However, these invasive open heart procedures in which the heart is stopped beating produce a high degree of trauma, a significant risk of complications, an extended hospital stay, and a painful recovery period for the patient. Moreover, while heart valve surgery produces beneficial results for many patients, numerous others who might benefit from such surgery are unable or unwilling to undergo the trauma and risks of such open heart techniques.

Techniques for minimally invasive thoracoscopic repair of heart valves while the heart is still beating have also been developed. U.S. Pat. No. 8,465,500 to Speziali, which is incorporated by reference herein, discloses a thoracoscopic heart valve repair method and apparatus. Instead of requiring open heart surgery on a stopped heart, the thoracoscopic heart valve repair methods and apparatus taught by Speziali utilize fiber optic technology in conjunction with transesophageal echocardiography (TEE) as a visualization technique during a minimally invasive surgical procedure that can be utilized on a beating heart. More recent versions of these techniques are disclosed in U.S. Pat. Nos. 8,758,393 and 9,192,374 to Zentgraf, which are also incorporated by reference herein and disclose an integrated device that can enter the heart chamber, navigate to the leaflet, capture the leaflet, confirm proper capture, and deliver a suture as part of a mitral valve regurgitation (MR) repair. In some procedures, these minimally invasive repairs are generally performed through a small, between the ribs access point followed by a puncture into the ventricle through the apex of the heart. Although far less invasive and risky for the patient than an open heart procedure, these procedures still require significant recovery time and pain.

Some systems have therefore been proposed that utilize a catheter routed through the patient's vasculature to enter the heart and attach a suture to a heart valve leaflet as an artificial chordae. While generally less invasive than the approaches discussed above, transcatheter heart valve repair can provide additional challenges. For example, with all artificial chordae replacement procedures, in addition to inserting a suture through a leaflet, the suture must also be anchored at a second location, such as at a papillary muscle in the heart, with a suture length, and tension and positioning of the suture should be adjustable to enable the valve to function naturally. If the suture is too short and/or has too much tension, the valve leaflets may not properly close. Conversely, if the suture is too long and/or does not have enough tension, the valve leaflets may still be subject to prolapse. Proper and secure anchoring of the suture at the second position away from the leaflet is therefore a critical aspect of any heart valve repair procedure for inserting an artificial chordae. In the case of transcatheter procedures, such anchoring can be difficult because it can be difficult for the flexible catheter required for routing through the patient's vasculature to apply sufficient force to stably insert traditional suture anchors into the heart wall, e.g., the myocardium.

### SUMMARY

Disclosed herein are embodiments of cardiac anchors configured to be inserted into a heart wall of a patient to anchor a suture as an artificial chordae under an appropriate tension for proper valve function. Such cardiac anchors are particularly suitable for use in intravascular, transcatheter procedures.

In an embodiment, an anchor assembly configured to be implanted into a heart wall of a heart of a patient to anchor a suture that is configured to extend from a valve leaflet of the heart as an artificial chordae can include an anchor hub having a proximal drive socket configured to interface with a delivery tool and defining an outer clamp surface and a helical coil extending distally from the anchor hub and having a sharpened tip configured to embed the helical coil into the heart wall upon rotation of the helical coil caused by the delivery tool interfacing with the drive socket. A spring crimp can have an open position and a closed position with the crimp biased towards the closed position. A locking element can be configured to interface with the spring crimp to selectively hold the spring crimp in the open position. Removal of the locking element from the spring crimp with the spring crimp positioned over the anchor hub can cause the spring crimp to clamp onto the outer clamp surface of the anchor hub with sufficient force to hold a suture between the spring crimp and the outer clamp surface under tension.

In an embodiment, an anchor assembly configured to be implanted into a heart wall of a heart of a patient to anchor a suture that is configured to extend from a valve leaflet of the heart as an artificial chordae can include an anchor configured to be inserted into a heart wall of a patient. A spring crimp can have an open position and a closed position with the crimp biased towards the closed position. A locking element can be configured to interface with the spring crimp to selectively hold the spring crimp in the open position. Removal of the locking element from the spring crimp with the spring crimp positioned over the anchor hub can cause the spring crimp to clamp onto the outer clamp surface of the anchor hub with sufficient force to hold a suture between the spring crimp and the outer clamp surface under tension.

In an embodiment, an anchor assembly can be configured to be implanted into a heart wall of a heart of a patient to anchor a suture that is configured to extend from a valve leaflet of the heart as an artificial chordae. The anchor assembly can include a spring crimp having an open position and a closed position with the spring crimp being biased towards the closed position and including one or more locking rings. A locking element can be configured to be inserted through the one or more locking rings of the spring crimp to selectively hold the spring crimp in the open position, wherein removal of the locking rod from the one or more locking rings of the spring crimp with the spring crimp positioned over a suture anchor causes the spring crimp to clamp onto the outer clamp surface of the suture anchor with sufficient force to hold a suture between the spring crimp and the suture anchor under tension as an artificial chordae extending from the valve leaflet to the suture anchor.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a schematic representation of a method for inserting a leaflet capture catheter into a beating heart of a patient according to an embodiment.

FIG. 2 depicts a suture anchor according to an embodiment.

FIGS. 3A-3D depict a suture clip according to an embodiment.

FIGS. 4A-4M schematically depict a procedure for anchoring an artificial chordae according to an embodiment.

FIG. 5 depicts a flowchart of method steps for a heart valve repair procedure according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure is generally directed to inserting and anchoring one or more sutures as artificial chordae into one or more heart valve leaflets through an intravascular, transcatheter approach. A heart valve leaflet may be captured and a suture inserted through the leaflet in any manner known in the art. Examples of such leaflet capture catheters are disclosed in copending U.S. Patent Publication No. 2019/0290260 and U.S. patent application Ser. No. 16/564,887, each of which is hereby incorporated by reference herein. Other transcatheter procedures for inserting an artificial chordae are disclosed in U.S. Patent Publication No. 2016/0143737 and U.S. patent application Ser. No. 16/745,074, each of which is hereby incorporated by reference herein. In other embodiments, artificial chordae can be inserted through transapical, open, or any other approach.

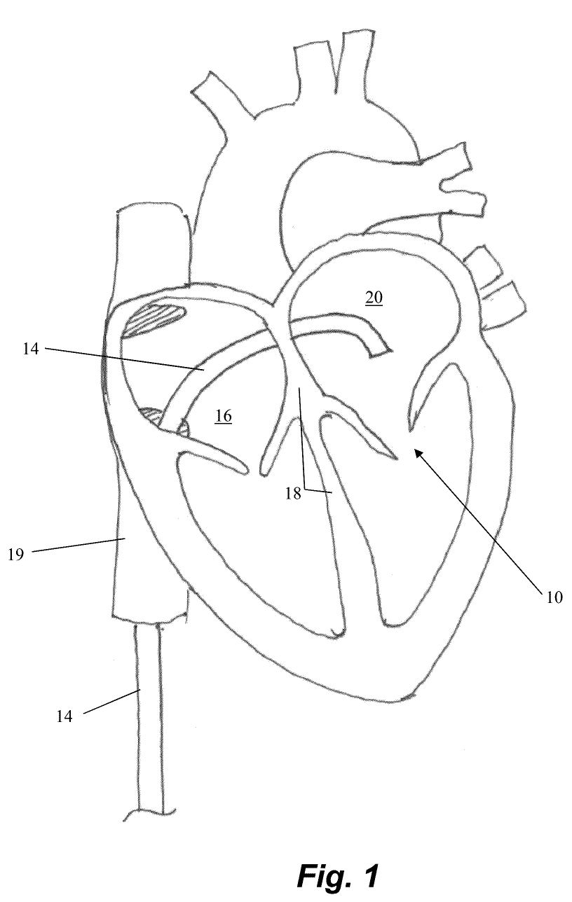

In embodiments, access into the heart to the valve being repaired can be gained through an intravascular, transcatheter approach. If the valve being repaired is the mitral valve, the valve may further be accessed transseptally. FIG. 1 depicts a schematic representation of an embodiment of an access approach for a heart valve repair system accessing the mitral valve 10. FIG. 1 depicts a guide catheter 14 accessing the interior of the heart via the femoral vein. In some embodiments, such a system can further include an outer guide catheter and an inner guide catheter. In such embodiments, the outer guide catheter can be inserted into the femoral vein at the patient's groin and advanced through the femoral vein into the inferior vena cava 19 and then into the right atrium 16. In various embodiments, the outer guide catheter can be steerable in a single plane and can have an outer diameter of about or less than about 30 French, such as, for example 24 French. The septum 18 can then be punctured using an appropriate puncture tool and the outer guide catheter advanced into the septum 18 or through the septum 18 into the left atrium 20. The inner guide catheter can then be axially advanced through the outer guide catheter into the left atrium 20. In some embodiments, the inner guide catheter can have two planes of steerability and can be maneuvered along with and/or beyond the outer guide catheter to establish a stable position superior to the mitral valve 10 and to provide a desired trajectory for operation of a leaflet capture catheter to repair the valve. In other embodiments, anchors as described herein may be implanted through other intravascular approaches as well as non-intravascular approaches.

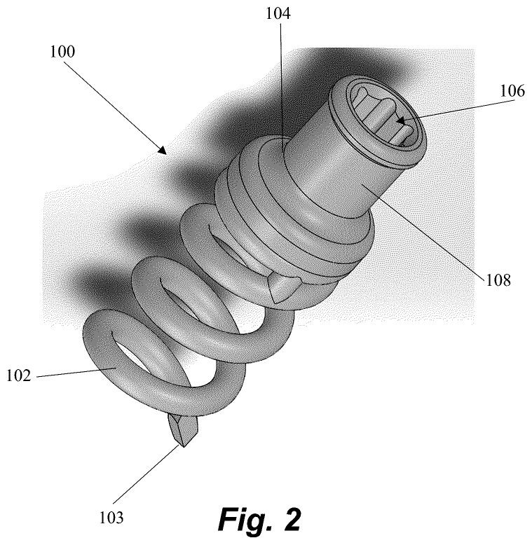

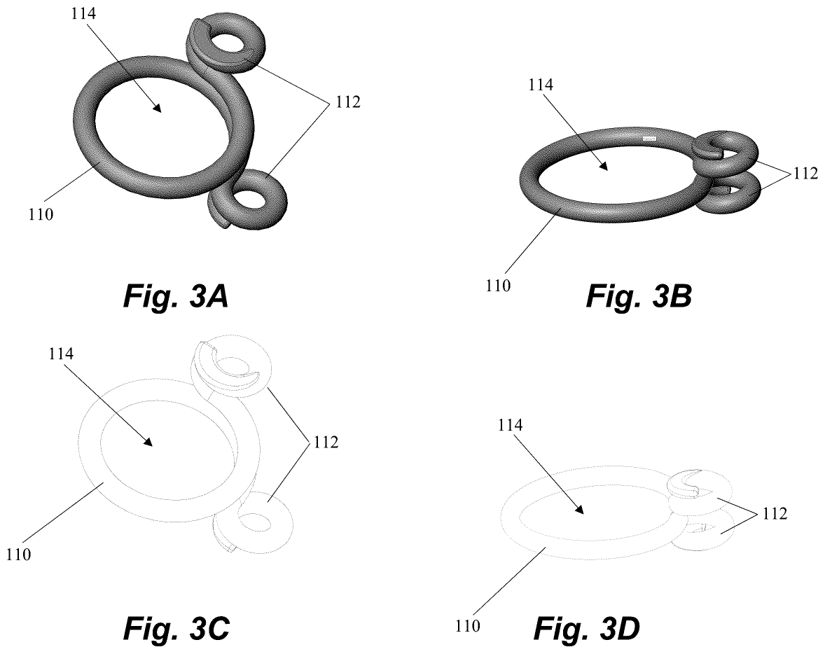

FIG. 2 depicts a suture anchor 100 for anchoring a suture as an artificial chordae in a heart wall of a patient. Anchor 100 includes an anchor coil 102 that embeds the anchor 100 into heart tissue and an anchor hub 104 that can be connected to the anchor coil 102. Anchor coil 102 can include a sharpened distal tip 103 to initially penetrate the tissue and one or more barbs (not pictured) to prevent the coil from unintentionally rotating back out of the tissue. Anchor hub 104 can include a drive socket 106 such, as e.g., a hexagonal drive, square drive etc. that interfaces with a drive tool used to rotate the anchor 100 to embed the anchor coil 102 into the heart tissue. The anchor hub 104 also defines an outer suture clamp surface 108 that in this configuration is generally cylindrical that is used to clamp sutures onto the anchor hub 104 as will be discussed in more detail below. FIGS. 3A-3D depict a suture spring crimp 110 that can be configured generally as a ring shown in both an open position (FIGS. 3B and 3D) and a closed position (FIGS. 3A and 3C). Spring crimp 110 crimp can include a pair of locking rings 112 configured to receive a locking pin. As will be described in more detail below, locking pin can be inserted through locking rings to hold the spring crimp 110 in the open position of FIGS. 3B and 3D for delivery to the anchor and then removed to release the crimp 110 into the closed position of FIGS. 3A and 3C to lock the spring crimp 110 and one or more sutures onto the anchor 100. In the open position the interior 114 of the crimp body of the spring crimp 110 therefore has a greater diameter than in the closed position, in which the interior 114 is sized to tightly engage the outer suture clamp surface 108 of the anchor hub 104.

In embodiments, the anchor coil 102 can be wound from a medical, implantable grade stainless steel wire, such as, for example 31LVM stainless steel, or other implantable grade, high-tensile metals. The diameter of the wire forming the coil 102 can be large enough to create an effectively rigid coil with respect to the tissue to allow the coil to be directed and turned into the tissue from the proximal end of the system, while still controlling the distal tip of the coil such that it will engage the tissue in a helical path as it is delivered. In embodiments, a wire diameter of 0.080″ with a pitch of 0.100″ along the coil enables the necessary control while enabling a sufficient amount of tissue to exist within the helical coil to ensure good engagement without disrupting the tissue to compromise the ability of the tissue to heal around the anchor or cause tissue necrosis. The total axial length of the coil can be sized to engage as much cardiac tissue as possible to provide secure anchoring while limiting the risk of perforation of the ventricle (i.e., the anchor extending completely through the heart wall). Average ventricular tissue thickness has been found to generally be within the range of 10-12 mm. In embodiments, a total length of the tissue engaging portion of the coil can be 7-9 mm to provide an appropriate balance of maximization of tissue engagement for secure anchoring while limiting risk of perforation.

Due to the complex contractile motion of cardiac tissue at any specific anchoring point within the myocardium, features to resist any anti-rotation of the of the coil back out of the myocardium after the anchor is implanted can be included such as one or more barbs attached to the anchor coil as noted above. In embodiments, the barb(s) can be safely attached to the coil by welding, such as, for example, by anchor welding. In embodiments, the barb can extend from about 0.025″ to 0.040″ outwardly from the profile of the coil and the tip of the barb can be bent proximally. Such a configuration reduces the insertion force with respect to the back-out force to provide more stability to the implanted anchor. The barb(s) can be oriented on the helical coil to reduce the potential of interaction between the barb(s) and a suture. As the coil is inserted, the suture is pressed, by the distal facing portions of the anchor coil, against the tissue as the coil is screwed into the tissue. The suture will remain at the tissue surface of the anchor site while the coil effectively passes by the suture leaving the suture trapped between the tissue surface and the distal surface of the coil as the coil is inserted. Because of this interaction, orientation and placement of the barb(s) is effected to keep the suture from interacting with the barb(s) which could result in potentially tangling or damage of the suture.

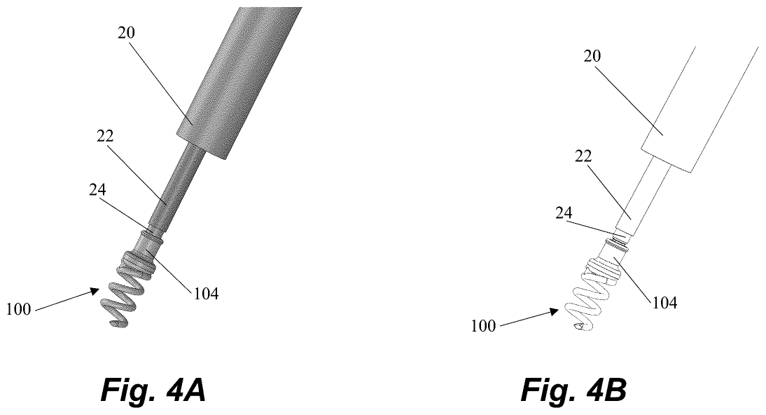

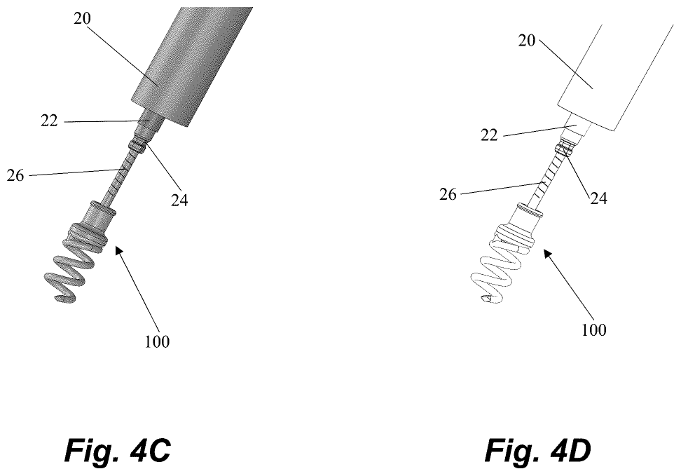

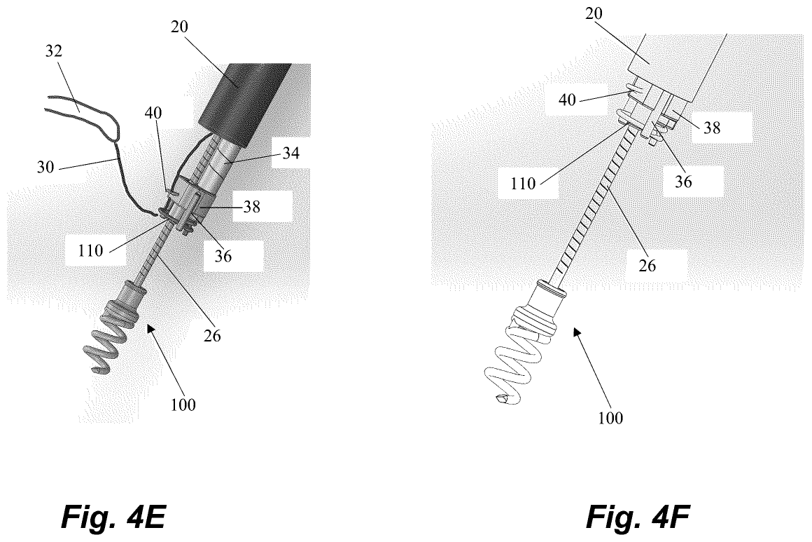

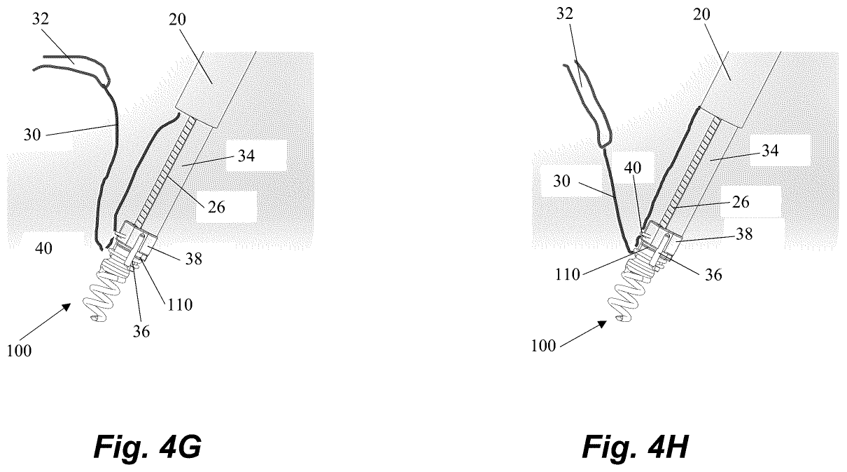

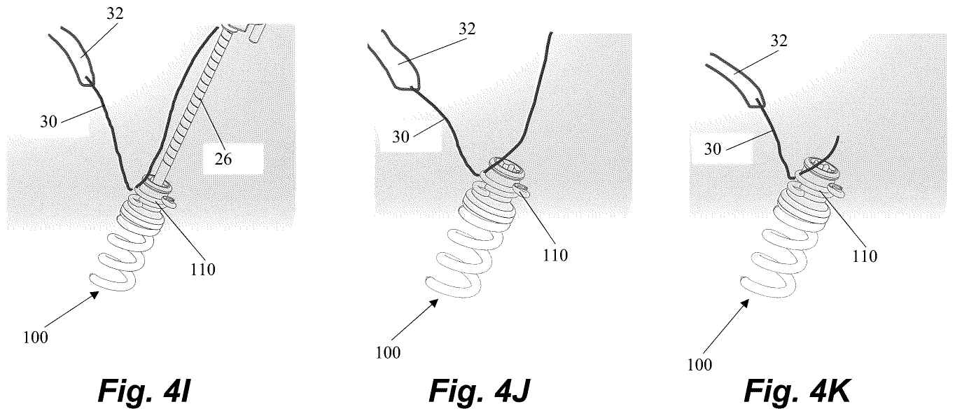

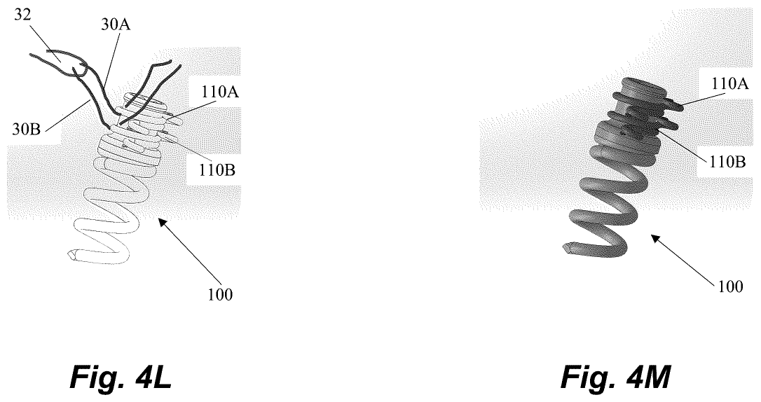

Referring now to FIGS. 4A-4M, schematic representations of a procedure for inserting an anchor into the heart such as anchor 100 described above is depicted. The heart can initially be accessed with a guide catheter 20 through various methods such as those described herein. An anchor driver 22 including a distal bit 24 shaped to engage with the drive socket 106 of the anchor hub 104 can be routed through the guide catheter 20 with the anchor 100 attached to the distal bit 24 as shown in FIGS. 4A-4B. In embodiments, the anchor driver 22 can be a flexible tube capable of being routed through the patient's vasculature through the guide catheter while also providing sufficient torque on the drive socket 106 to rotate the anchor 100 with sufficient force to embed the anchor into heart tissue. After the anchor 100 had been driven into tissue, the anchor driver 200 can be disconnected from the drive socket 106 and withdrawn back through the guide catheter 20 as shown in FIGS. 4C-4D. A tether 26 extending through the guide catheter 20 over which the anchor driver 22 was inserted can remain attached to the anchor 100 to provide access to the anchor 100 from outside the body. Tether 26 can be a flexible component that attaches to anchor 100 in any manner, such as by being rotationally attached (i.e. screwed into), laser welded, etc.

In some embodiments, one or more sutures 30 can have been previously inserted into a leaflet 32 prior to insertion of the anchor 100 into the heart tissue. In other embodiments, the anchor 100 can be inserted first and then one or more sutures inserted through a leaflet. Once the anchor 100 has been seated, a suture 30 can be threaded through the opening 114 in spring crimp 110 outside of the body. The spring crimp 110 can be attached to a delivery catheter 34 that is routed through guide catheter 20 into the heart. As can be seen in FIGS. 4E-4F, locking pin 36 can be inserted through the locking rings 112 of the spring crimp 110 to hold the crimp in the open position. Delivery catheter 34 can include a distal guide ring 38 having an aperture that can be inserted over tether 26 to guide the delivery catheter 34 to the anchor 100 in the heart and an eyelet 40 through which the suture is routed to retain the suture adjacent the delivery catheter.

Referring to FIGS. 4G-4H, the guide ring 38 of the delivery catheter 34 can be positioned adjacent to or over the anchor hub 104 of the anchor 100 such that the spring crimp 110 is positioned around the anchor hub 104. When the position of the spring crimp 110 is verified, the tension on the suture 30 can be adjusted for proper valve function. To lock the suture at the desired tension, the locking pin 36 can be pulled proximally to release the spring crimp 110 into the closed position around the anchor hub 104 locking the suture 30 between the spring crimp 110 and the outer suture clamp surface 108 of the anchor hub 104. In an embodiment, locking pin 36 can be a flexible elongate member that extends back through the delivery catheter 34 out of the body and can therefore be actuated at the proximal control handle outside of the body. As can be seen in, e.g., FIG. 4E, the spring crimp 110 is held distally of the delivery catheter 34 by locking pin 36 such that the crimp 110 can be positioned directly adjacent the suture clamping surface 108 of the anchor hub 104 and removal of the locking pin 36 from locking rings 112 releases the spring crimp 110 from the delivery catheter 34 onto the suture clamping surface 108. The delivery catheter 34 can then be withdrawn, the tether 26 unscrewed, cut, or otherwise disconnected from the anchor 100 and excess suture 30 can be cut adjacent the anchor 100. If additional sutures are desired to be anchored to the anchor 100, multiple suture crimps 110A, 110B may be inserted onto the anchor hub 104 to anchor multiple sutures 30A, 30B prior to removal of the tether 26 as shown in FIGS. 4L-4M.

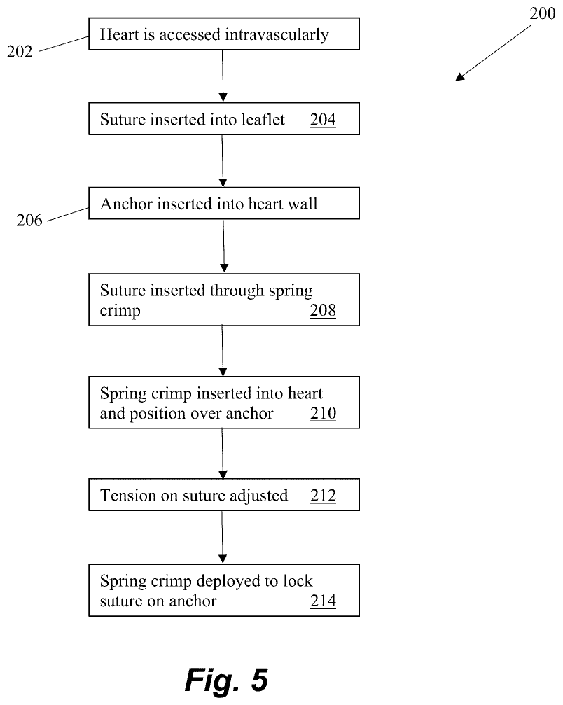

Referring now to FIG. 5, a flowchart of method steps for a heart valve repair procedure 200 is depicted. At step 202, the heart is accessed intravascularly and a suture is inserted into a valve leaflet at step 204. A suture anchor is inserted into the heart wall below the valve at step 206. The suture is inserted through a spring crimp outside of the body at step 208 and at step 210 the spring crimp is delivered into the heart and positioned over the suture anchor. The tension on the suture is adjusted for proper valve function at step 212. Once the desired tension is achieved, the spring crimp is deployed onto the anchor at step 214 to lock the suture at the desired tension.

In embodiments, an anchor assembly configured to be implanted into a heart wall of a heart of a patient to anchor a suture that is configured to extend from a valve leaflet of the heart as an artificial chordae can include an anchor hub having a proximal drive socket configured to interface with a delivery tool and defining an outer clamp surface and a helical coil extending distally from the anchor hub and having a sharpened tip configured to embed the helical coil into the heart wall upon rotation of the helical coil caused by the delivery tool interfacing with the drive socket. A spring crimp can have an open position and a closed position with the crimp biased towards the closed position. A locking element can be configured to interface with the spring crimp to selectively hold the spring crimp in the open position. Removal of the locking element from the spring crimp with the spring crimp positioned over the anchor hub can cause the spring crimp to clamp onto the outer clamp surface of the anchor hub with sufficient force to hold a suture between the spring crimp and the outer clamp surface under tension.

In some embodiments, the spring crimp comprises one or more locking rings, and the locking element interfaces with the one or more locking rings to hold the spring crimp in the open position.

In some embodiments, the spring crimp comprises a pair of locking rings that are axially aligned in the open position.

In some embodiments, the locking element is configured as an elongate rod.

In some embodiments, the elongate rod is inserted through one or more openings in the suture crimp to hold the suture crimp in the open position.

In some embodiments, the elongate rod is configured to be moved proximally relative to the anchor to be removed from the spring crimp.

In some embodiments, the elongate rod is configured to extend from the anchor inserted into the heart wall thorough the patient's vasculature and outside of the body.

In embodiments, an anchor assembly configured to be implanted into a heart wall of a heart of a patient to anchor a suture that is configured to extend from a valve leaflet of the heart as an artificial chordae can include an anchor configured to be inserted into a heart wall of a patient. A spring crimp can have an open position and a closed position with the crimp biased towards the closed position. A locking element can be configured to interface with the spring crimp to selectively hold the spring crimp in the open position. Removal of the locking element from the spring crimp with the spring crimp positioned over the anchor hub can cause the spring crimp to clamp onto the outer clamp surface of the anchor hub with sufficient force to hold a suture between the spring crimp and the outer clamp surface under tension.

In some embodiments, the spring crimp comprises one or more locking rings, and the locking element interfaces with the one or more locking rings to hold the spring crimp in the open position.

In some embodiments, the spring crimp comprises one or more locking rings, and the locking element interfaces with the one or more locking rings to hold the spring crimp in the open position.

In some embodiments, the spring crimp comprises one or more locking rings, and wherein the locking element interfaces with the one or more locking rings to hold the spring crimp in the open position.

In some embodiments, the spring crimp comprises one or more locking rings, and wherein the locking element interfaces with the one or more locking rings to hold the spring crimp in the open position.

In some embodiments, the spring crimp comprises a pair of locking rings that are axially aligned in the open position.

In some embodiments, the locking element is configured as an elongate rod.

In some embodiments, the elongate rod is inserted through one or more openings in the suture crimp to hold the suture crimp in the open position.

In some embodiments, the elongate rod is configured to be moved proximally relative to the anchor to be removed from the spring crimp.

In some embodiments, the elongate rod is configured to extend from the anchor inserted into the heart wall thorough the patient's vasculature and outside of the body.

In embodiments, an anchor assembly can be configured to be implanted into a heart wall of a heart of a patient to anchor a suture that is configured to extend from a valve leaflet of the heart as an artificial chordae. The anchor assembly can include a spring crimp having an open position and a closed position with the spring crimp being biased towards the closed position and including one or more locking rings. A locking element can be configured to be inserted through the one or more locking rings of the spring crimp to selectively hold the spring crimp in the open position, wherein removal of the locking rod from the one or more locking rings of the spring crimp with the spring crimp positioned over a suture anchor causes the spring crimp to clamp onto the outer clamp surface of the suture anchor with sufficient force to hold a suture between the spring crimp and the suture anchor under tension as an artificial chordae extending from the valve leaflet to the suture anchor.

In some embodiments, the spring crimp comprises a pair of locking rings that are axially aligned in the open position.

In some embodiments, the locking element is configured as an elongate rod.

In some embodiments, the elongate rod is inserted through one or more openings in the suture crimp to hold the suture crimp in the open position.

In some embodiments, the elongate rod is configured to be moved proximally relative to the anchor to be removed from the spring crimp.

In some embodiments, the elongate rod is configured to extend from the anchor inserted into the heart wall thorough the patient's vasculature and outside of the body.

Various other anchors can be interchangeably employed in each of the above-described systems. Such anchors can include those disclosed in U.S. Patent Application Publication Nos. 2019/0343626; 2019/0343633; 2019/0343634; 2020/0330228; and 2021/0220138, which are hereby incorporated by reference.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(1) are not to be invoked unless the specific terms “means for” or “step for” are recited in a claim.

1. An anchor assembly configured to be implanted into a heart wall of a heart of a patient to anchor a suture that is configured to extend from a valve leaflet of the heart as an artificial chordae, the anchor assembly comprising:
an anchor hub having a proximal drive socket configured to interface with a delivery tool and defining an outer clamp surface;
a helical coil extending distally from the anchor hub and having a sharpened tip configured to embed the helical coil into the heart wall upon rotation of the helical coil caused by the delivery tool interfacing with the drive socket;
a spring crimp having an open position and a closed position, the spring crimp being biased towards the closed position; and
a locking element configured to interface with the spring crimp to selectively hold the spring crimp in the open position, wherein removal of the locking element from the spring crimp with the spring crimp positioned over the anchor hub causes the spring crimp to clamp onto the outer clamp surface of the anchor hub with sufficient force to hold a suture between the spring crimp and the outer clamp surface under tension as an artificial chordae extending from the valve leaflet to the anchor.

2. The anchor assembly of claim 1, wherein the spring crimp comprises one or more locking rings, and wherein the locking element interfaces with the one or more locking rings to hold the spring crimp in the open position.

3. The anchor assembly of claim 2, wherein the spring crimp comprises a pair of locking rings that are axially aligned in the open position.

4. The anchor assembly of claim 1, wherein the locking element is configured as an elongate rod.

5. The anchor assembly of claim 4, wherein the elongate rod is inserted through one or more openings in the suture crimp to hold the suture crimp in the open position.

6. The anchor assembly of claim 4, wherein the elongate rod is configured to be moved proximally relative to the anchor to be removed from the spring crimp.

7. The anchor assembly of claim 4, wherein the elongate rod is configured to extend from the anchor inserted into the heart wall thorough the patient's vasculature and outside of the body.

8. An anchor assembly configured to be implanted into a heart wall of a heart of a patient to anchor a suture that is configured to extend from a valve leaflet of the heart as an artificial chordae, the anchor assembly comprising:
an anchor configured to be inserted into a heart wall of a patient;
a spring crimp having an open position and a closed position, the spring crimp being biased towards the closed position; and
a locking element configured to selectively hold the spring crimp in the open position, wherein removal of the locking element from the spring crimp with the spring crimp positioned over the anchor causes the spring crimp to clamp onto the anchor with sufficient force to hold a suture between the spring crimp and the anchor under tension as an artificial chordae extending from the valve leaflet to the anchor.

9. The anchor assembly of claim 8, wherein the spring crimp comprises one or more locking rings, and wherein the locking element interfaces with the one or more locking rings to hold the spring crimp in the open position.

10. The anchor assembly of claim 9, wherein the spring crimp comprises a pair of locking rings that are axially aligned in the open position.

11. The anchor assembly of claim 8, wherein the locking element is configured as an elongate rod.

12. The anchor assembly of claim, 11 wherein the elongate rod is inserted through one or more openings in the suture crimp to hold the suture crimp in the open position.

13. The anchor assembly of claim 11, wherein the elongate rod is configured to be moved proximally relative to the anchor to be removed from the spring crimp.

14. The anchor assembly of claim 11, wherein the elongate rod is configured to extend from the anchor inserted into the heart wall thorough the patient's vasculature and outside of the body.

15. An anchor assembly configured to be implanted into a heart wall of a heart of a patient to anchor a suture that is configured to extend from a valve leaflet of the heart as an artificial chordae, the anchor assembly comprising:
a spring crimp having an open position and a closed position, the spring crimp being biased towards the closed position and including one or more locking rings; and
a locking element configured to be inserted through the one or more locking rings of the spring crimp to selectively hold the spring crimp in the open position, wherein removal of the locking rod from the one or more locking rings of the spring crimp with the spring crimp positioned over a suture anchor causes the spring crimp to clamp onto the outer clamp surface of the suture anchor with sufficient force to hold a suture between the spring crimp and the suture anchor under tension as an artificial chordae extending from the valve leaflet to the suture anchor.

16. The anchor assembly of claim 15, wherein the spring crimp comprises a pair of locking rings that are axially aligned in the open position.

17. The anchor assembly of claim 15, wherein the locking element is configured as an elongate rod.

18. The anchor assembly of claim 17, wherein the elongate rod is inserted through one or more openings in the suture crimp to hold the suture crimp in the open position.

19. The anchor assembly of claim 17, wherein the elongate rod is configured to be moved proximally relative to the anchor to be removed from the spring crimp.

20. The anchor assembly of claim 17, wherein the elongate rod is configured to extend from the anchor inserted into the heart wall thorough the patient's vasculature and outside of the body.
